(19) United States
(12) Patent Application Publication
(10) Pub. No.: US 2007/0162062 A1
(43) Pub. Date: Jul. 12, 2007

(54) Reciprocating apparatus and methods for removal of intervertebral disc tissues

(76) Inventors: Britt K. Norton, Eden Prairie, MN (US); Christine M. Horton, Prior Lake, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/297,603

(22) Filed: Dec. 8, 2005

(51) Int. Cl. A61B 17/32

(52) U.S. Cl. 606/170

(57) ABSTRACT

Reciprocating cutting apparatus and methods for removing tissue from an intervertebral disc are disclosed. The reciprocating cutting apparatus can include a guide tube, a cutting cap and a drive shaft. Other reciprocating cutting apparatus can include a guide tube, an outer guide tube, a cutting cap and a drive shaft. The cutting cap reciprocates relative to the distal opening in the guide tube to cut and/or abrade tissues within an intervertebral disc. A cutting member may be provided to assist in the cutting and/or abrading of the tissues within the intervertebral disc. The reciprocating cutting apparatus may be generally configured to extend and withdraw a guide tube from and into the distal opening in the outer guide tube.

### BACKGROUND OF THE INVENTION

1. Field of the Invention

The present invention relates to removal of intervertebral discs and, more particularly, to apparatus and methods for removal of the nucleus pulposus of an intervertebral disc.

2. Description of the Related Art

The spine is made up of twenty-four bony vertebrae, each separated by a disc that both connects the vertebrae and provides cushioning between them. The lumbar portion of the spine has five vertebrae, the last of which connects to the sacrum. The disc is comprised of the annulus fibrosus, which is a tough, layered ligamentous ring of tissue that connects the vertebrae together, and the nucleus, a gelatinous material that absorbs water and nutrients through the endplates of the vertebrae. In a healthy disc, the nucleus pulposus is pressurized within the annulus much like the air is pressurized within an automobile tire.

Degenerative disc disease (DDD) is a condition that affects both the annulus fibrosus and nucleus pulposus of the disc, and is usually thought of as a cascade of events. In general, DDD is characterized by a weakening of the annulus and permanent changes in the nucleus, and may be caused by extreme stresses on the spine, poor tone of the surrounding muscles, poor nutrition, smoking, or other factors. In DDD, the nutrient flow to the nucleus is disrupted and the nucleus loses water content. As the nucleus dehydrates it loses pressure, resulting in a loss of disc height and a loss in the stability of that segment of the spine. In the lumbar spine, as the degenerative cascade continues, the annulus may bulge and press on a nerve root, causing sciatica (leg pain) among other problems. The loss of disc height can also result in leg pain by reducing the size of the opening for the nerve root through the bony structures of the spine. As the disc loses height, the layers of the annulus can begin to separate, irritating the nerves in the annulus and resulting in back pain.

Surgical treatment for early DDD, where the pain is primarily leg pain, is usually a discectomy where some of the nucleus material is removed to reduce the bulging of the disc and the pressure on the nerve root. For more severe cases of DDD, where the disc has completely collapsed and/or where a discectomy did not have long-term success, the traditional surgical treatment has been fusion of the vertebrae through the use of plates, rods, pedical screws, and interbody fusion devices. For years, surgeons and industry have been looking for ways to interrupt the degenerative cascade for patients with early stage disease, and for methods that retain motion at the affected disc in patients with more advanced disease. Just as the surgical treatment for degenerated knees and hips changed from fusion to motion preservation (arthrodesis to arthroplasty), innovative technologies are now creating a market for treatment of DDD without resorting to fusion. The field of spinal arthroplasty represents a significant emerging market in spinal surgery.

Surgical treatment for early stage disease that involves primarily leg pain as a result of a herniated disc is currently limited to a simple discectomy, where a small portion of the disc nucleus is removed to reduce pressure on the nerve root, the cause of the leg pain. While this procedure is usually immediately successful, it offers no means to prevent further degeneration, and a subsequent herniation requiring surgery will occur in about 15% of these patients.

A range of prosthetic techniques has been developed and continues to be developed for the treatment of DDD. These techniques typically use one of three types of prosthetic devices: total disc replacement (TDR) devices, which sacrifice much of the connective tissue of the disc and are intended for discs with severe degeneration; partial disc replacement (PDR) devices, which replace only the nucleus of the disc; and flexible springs and connectors attached to the posterior bony elements of the spine. The PDR will be marketed as the surgical treatment of choice for patients with slightly more advanced (mild-to-moderate) disc degeneration. This technology relies on the connective structures of the affected level, such as the annulus, facets, and longitudinal ligaments, to be relatively healthy. A fourth type of device, used for repairing the annulus after a herniation or implantation of a PDR, is also currently in development.

Current designs for nucleus replacement devices are typically not attached to the nucleus or vertebra, and are free to move within the nucleus cavity. Much like the healthy nucleus, these devices are subjected to the high forces and the twisting and bending motions that must be endured by the spinal structures, and some device movement is expected. Current PDR devices have a known complication of excessive device movement, however, and can move back out the annulus at the site of implantation. This device extrusion can occur in over 25% of cases for some designs. While the effect of the complication is not life threatening, the response is another surgery to reposition or replace the PDR, or to remove it altogether and likely replace it with a total disc replacement or a fusion procedure. There is mounting evidence that the nucleus material left in the disc cavity, even after an exhaustive removal procedure, can push against even a well-positioned PDR and be the cause of many of the device extrusions. When a posterior approach is used for removal, the remaining nucleus material left behind can push against a PDR. While more of this material could be removed if the disc is accessed via a lateral or an anterior approach, current information indicates that most spine surgeons prefer to use the posterior approach.

The annulus repair technologies that rely on mechanical means to close the annulus involve the need to contact and/or secure to the inside of the annulus tissue immediately adjacent to the site used to access the nucleus cavity. These designs will achieve the best deployment and surgical attachment to the annulus if the bulk of the relatively soft nucleus material near the access site has been adequately removed. Remaining nucleus material can have a negative impact on the performance of these devices if it is not removed. This material will be difficult to remove whether the access to the cavity is performed via a posterior, lateral, or an anterior surgical approach.

For annulus repair and PDR, among other procedures, implantation site preparation typically involves removal of the nucleus. A wide range of devices have been developed for this removal procedure. However, surgeons have historically utilized an array of pituitary rongeurs for the various procedures requiring removal of the nucleus pulposus or portions of the nucleus pulposus.

The rongeur is provided in a variety of configurations including “up-biting”; straight; and “down-biting”, and can be found in a variety of lengths, widths, and with razor or serrated jaws. However, even using the preferred posterior access to the disc with a rongeur, its useful range of motion within the intervertebral disc is limited. The bony structure of the posterior spinal elements, even though partially removed to provide access for PDR implantation, typically limits the angles through which the rongeur can be maneuvered. This limitation of movement serves to limit the amount of nucleus material that can be removed. More importantly, the limitation on movement may not allow adequate removal of material next to the annular access to provide good contact for an annular repair device and does not allow adequate removal of material contralateral to the annular access, preventing optimal placement for a PDR. Further, the use of a rongeur requires constant insertion and removal to clean the nucleus material from the tip of the device, resulting in dozens of insertion/removal steps to remove an adequate amount of material from the nucleus. This can increase the trauma to the surrounding annulus tissue and increase the risk of damaging the endplates.

An additional significant limitation of the rongeur instrument is the ability to easily remove the important annular tissue, especially when using rongeurs with a sharp cutting tip. Surgeons typically do not try to remove the entire nucleus in simple discectomy procedures, or intentionally remove annulus in preparation for fusion procedures. In this respect, a surgeon's “feel” for the tissue, or ability to distinguish softer nucleus tissue from tougher annulus tissue, may not be well developed and PDR site preparation may result in significant trauma to the annulus.

A range of more sophisticated devices for removing nucleus has been developed; however, the adoption of these devices has been very limited. Some of the more intricate devices utilize mechanized cutting mechanisms for removal of material from the nucleus pulposus. Frequently, these devices require suction and/or irrigation to remove material during the procedure.

One device uses a guillotine-style assembly that cuts nucleus material, aspirates the material into the instrument tip, and then evacuates the cut material is through the instrument. Movement of the guillotine assembly is automated and controlled by a mechanism in the handpiece of the instrument. The continuous removal of tissue without the need to repeatedly insert and remove the instrument minimizes trauma to the surrounding tissue. The guillotine type assembly is typically associated with a straight, stiff device that is intended for a minimally invasive, percutaneous approach. Because of their stiffness, although the devices may be somewhat effective for a lateral or anterior surgical approach for PDR implantation, they are generally not usable for nucleus removal utilizing a posterior approach.

Other devices have utilized an Archimedes type screw to pull nucleus material into the catheter and shear it when it reaches the tip of the catheter. Continued collection of nucleus material by the rotating Archimedes type screw pushes the sheared material through the catheter and into a collection chamber. While less complicated to use than the previously discussed guillotine type assembly, the devices utilizing the Archimedes type screw typically have the similar maneuverability disadvantages. Further, these devices can relatively easily be directed into and through the annulus of the intervertebral disc being treated.

Still other systems have used a high-pressure stream of water to remove nucleus material. In one device, the high-pressure stream of water produces a vacuum which pulls nucleus material into the stream. The high-pressure stream of water then cuts the nucleus material and pulls the material through a catheter to a collection bottle. Among other disadvantages, such systems are expensive. Further, although the tip of the instrument can be bent slightly, its lateral reach when used via the posterior approach is still very limited. Further, since the water stream is very narrow, successful nucleus removal can be technique dependent and time consuming.

Still other devices utilize radio frequency (RF) energy or plasma directed through electrodes for tissue resection and vessel cauterization in preparation for implanting a PDR. These devices typically include an RF generator that can be used with a variety of different types and shapes of electrodes. These devices are typically stiff and have little lateral reach when used making them relatively ineffective for use through the posterior approach. Further, the RF ablation technology can resect annulus or endplate cartilage as easily as nucleus material.

Still other devices utilize lasers to remove material from the nucleus pulposus. These lasers are typically transmitted through a laser fiber positioned within a multi-lumen catheter. These multi-lumen catheters have also included additional components such as imaging fibers, illumination fibers, and irrigation ports. Further, the tip of these catheters can be steerable. Although steerable, the bend radius of the catheters typically prevents them from being useful for removing nucleus near the annulus access. Accordingly, these devices have limited utility for removal of material in preparation for implantation of annulus repair devices. Further, the effective radius of the laser beam from these devices is typically only 0.5 mm, making removal of large amounts of nucleus very difficult and time consuming. Detrimentally, lasers can resect annulus or endplate cartilage as easily as nucleus material. Since the tip of the catheter is typically not protected, the laser beam has the ability to easily penetrate and damage the annulus and endplate tissue.

Other devices for nucleus removal are also available. However, these technologies possess their own limitations for the unique needs of annulus repair and PDR device site preparation. The limitations of these devices, along with those of the pituitary rongeur, are driving the need for a more advanced instrument for nucleus removal.

### SUMMARY OF THE INVENTION

Apparatus and methods in accordance with the present invention may resolve many of the needs and shortcomings discussed above and will provide additional improvements and advantages as will be recognized by those skilled in the art upon review of the present disclosure.

In one aspect, the present invention may provide a reciprocating cutting apparatus for removing tissue from an intervertebral disc. The reciprocating cutting apparatus may include a guide tube, a drive shaft and a cutting cap. The guide tube may define a lumen extending through the guide tube from a proximal opening at a proximal end of the guide tube to a distal opening at a distal end of the guide tube. The lumen may include a bend at the distal end of the guide tube. Typically, the lumen of the guide tube extends linearly over a linear section extending between the bend and the distal opening. The guide tube may be slidably received within an outer guide tube. The cutting cap is typically movable between an extended position and a retracted position relative to the distal opening of the guide tube. The cutting cap has a trailing cutting edge and an atraumatic crown. The guide tube may include a cutting surface on a distal end of the guide tube to receive the trailing cutting edge of the cutting cap when the cutting cap is in a withdrawn position. The cutting surface may be defined on a cutting member secured to or within the distal end of the guide tube. The cutting member may be secured within the lumen of the guide tube at the distal opening of the lumen. The drive shaft has a proximal end and a distal end. The drive shaft may be received within the lumen of the guide tube. The drive shaft is operably connected to the cutting cap to confer a reciprocating motion to the cutting cap. The drive shaft may be operably connected to the cutting cap through a cam and cam follower system, through a direct mechanical connection, or through other indirect mechanisms for operably connecting the drive shaft to the cutting cap to confer a reciprocating motion to the cutting cap. The cutting cap may be secured directly to the drive shaft. When a cam and cam follower system is used to operably connect the drive shaft to the cutting cap, the cam system may include a cam, a cam follower and a cap shaft. The cam may be rotatably secured within the lumen of the guide tube. The cam defines a cam surface to slidably receive the cam follower. The cam may also include a shaft mount to secure the drive shaft to the cam. The cam follower may be biased against the cam surface to convert a rotation of the cam into a reciprocating motion. A spring may be used to bias the cam follower against the cam surface. The cap shaft may be secured at a first end of the cap shaft to the cam follower and at a second end of the cap shaft to the cutting cap. The reciprocating cutting apparatus may further include a motor connected to a distal end of the drive shaft to confer a reciprocating motion or rotational motion to the drive shaft. The motor may be slidably secured within a housing. A distal stop may be secured to the distal end of the guide tube. The distal stop may be secured to the guide tube by one or more stop supports. The stop supports may extend between the distal end of the guide tube and the distal stop to secure the distal stop relative to the distal opening of the guide tube. The cutting cap may include one or more cap guides secured to the cutting cap and slidably receiving at least one of the stop supports. The cap guides may be integral with the cutting cap.

### BRIEF DESCRIPTION OF THE DRAWINGS

FIG. 1 illustrates a perspective view of an embodiment of an apparatus in accordance with the present invention;

FIG. 2A illustrates an end side view of the distal portion of an embodiment of an apparatus in accordance with the present invention;

FIG. 2B illustrates a partial side view of the distal portion of the embodiment of the apparatus similar to the embodiment shown in FIG. 2A;

FIG. 2C illustrates a partial perspective view of the distal portion of the embodiment of the apparatus similar to the embodiment shown in FIG. 2A;

FIG. 3 illustrates a cross-section of an exemplary embodiment of the distal portion of an apparatus in accordance with the present invention in which the cutting cap cuts against the distal end of the guide tube;

FIG. 4A illustrates a cross-section of an exemplary embodiment of the distal portion of an apparatus in accordance with the present invention with the cutting cap in an at least partially extended position;

FIG. 4B illustrates a cross-section of an exemplary embodiment of the distal portion of an apparatus in accordance with the present invention with the cutting cap in a withdrawn position;

FIG. 5A illustrates a cross-section of another exemplary embodiment of the distal portion of an apparatus in accordance with the present invention with the cutting cap in an at least partially extended position;

FIG. 5B illustrates a cross-section of another exemplary embodiment of the distal portion of an apparatus in accordance with the present invention with the cutting cap in a withdrawn position;

FIG. 5C illustrates an end view of the embodiment of FIG. 5C through section lines 5C-5C;

FIG. 5D illustrates an end view of the embodiment of FIG. 5A through section lines 5D-5D;

FIG. 6 illustrates an end view of an exemplary embodiment of a cam in accordance with the present invention;

FIG. 7A illustrates a cross-section of yet another exemplary embodiment of the distal portion of an apparatus in accordance with the present invention with the cutting cap in a withdrawn position;

FIG. 7B illustrates an end side view of the distal portion of the embodiment of the apparatus shown in FIG. 7A;

FIG. 7C illustrates a sectioned view of the distal portion illustrating some details of the embodiment of the apparatus shown in FIG. 7A;

FIG. 8A illustrates a cross-section of still yet another exemplary embodiment of the distal portion of an apparatus in accordance with the present invention with the cutting cap in a withdrawn position;

FIG. 8B illustrates a cross-section of still yet another exemplary embodiment of the distal portion of an apparatus in accordance with the present invention with the cutting cap in an extended position;

FIG. 9A illustrates a cross-section of an exemplary embodiment of a distal portion of an apparatus in accordance with the present invention with the cutting cap in an at least partially extended position;

FIG. 9B illustrates a cross-section of another exemplary embodiment of a distal portion of an apparatus in accordance with the present invention with the cutting cap in an at least partially extended position;

FIG. 9C illustrates a cross-section of another exemplary embodiment of a distal portion of an apparatus in accordance with the present invention with the cutting cap in an at least partially extended position;

FIG. 9D illustrates a cross-section of another exemplary embodiment of a distal portion of an apparatus in accordance with the present invention with the cutting cap in an at least partially extended position; and

FIGS. 10A, 10B and 10C illustrate a sequential series of top views of aspects of an exemplary embodiment of an apparatus in accordance with the present invention advancing through the nucleus pulposus of an intervertebral disc.

All Figures are illustrated for ease of explanation of the basic teachings of the present invention only; the extensions of the Figures with respect to number, position, relationship and dimensions of the parts to form the preferred embodiment will be explained or will be within the skill of the art after the following description has been read and understood. Further, the exact dimensions and dimensional proportions to conform to specific force, weight, strength, and similar requirements will likewise be within the skill of the art after the following description has been read and understood.

Where used in various Figures of the drawings, the same numerals designate the same or similar parts. Furthermore, when the terms “top,” “bottom,” “right,” “left,” “forward,” “rear,” “first,” “second,” “inside,” “outside,” and similar terms are used, the terms should be understood to reference only the structure shown in the drawings as it would appear to a person viewing the drawings and utilized only to facilitate describing the illustrated embodiment.

### DETAILED DESCRIPTION OF THE INVENTION

The present invention provides a reciprocating cutting apparatus 10 and methods for removal of materials from an intervertebral disc positioned between adjacent vertebral bodies within the spine of a patient. Reciprocating cutting apparatus 10 in accordance with the present invention generally include a guide tube 12, and a cutting cap 14 as are illustrated generally throughout the figures for exemplary purposes. In some embodiments, reciprocating cutting apparatus 10 may also include a cutting member 16. The cutting cap 14 can reciprocate relative to the cutting member 16 to generate a cutting and/or abrading action to remove tissue from the nucleus pulposus of an intervertebral disc. In one aspect, the reciprocating cutting apparatus 10 may provide a cutting cap 14 and cutting member 16 which are extendable from the distal tip of an outer guide tube 20 for accessing tissues of an intervertebral disc. When extendable, the reciprocating cutting apparatus 10 may permit access to tissues remote from the distal end of the outer guide tube 20 positioned at a desired location within a patient. The reciprocating cutting apparatus 10 is typically configured to permit access to the intervertebral disc in a minimally invasive manner. In another aspect, the cutting cap 14 and cutting member 16 are retractable into the distal end of the outer guide tube 20 to better facilitate insertion and/or removal of the distal end of guide tube 20 from the intervertebral disc of a patient. In another aspect, the cutting cap 14 and cutting member 16 are configured to extend from and retract into the outer guide tube 20 while cutting cap 14 reciprocates relative to the cutting member 16 to remove or facilitate the removal of tissue from the intervertebral disc. The reciprocating cutting apparatus 10 may be generally configured to permit posterior access to the intervertebral disc wherein guide tube 12 may have sufficient flexibility to bend around various anatomical features and structures of the spine.

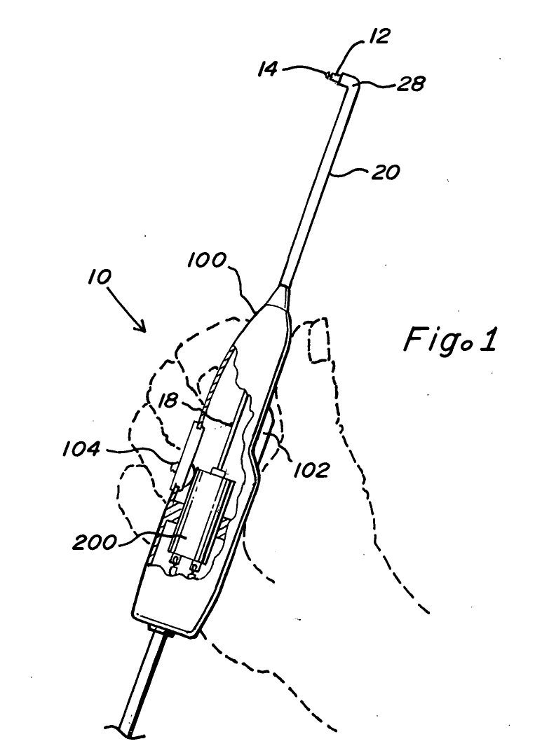

FIG. 1 illustrates an exemplary embodiment of a reciprocating cutting apparatus 10 in accordance with the present invention including a housing 100 and motor 200. The reciprocating cutting apparatus 10 may include a guide tube 12 having a bend 28 adjacent the distal end of guide tube 12 to direct a cutting cap 14 and cutting member 16 laterally. In other embodiments, the guide tube 12 may be flexible and received in a lumen 34 of an outer guide tube 20. A drive shaft 18 extends through the guide tube 12 to confer a reciprocating motion to cutting cap 14. As illustrated, the drive shaft 18 is connected to the motor 200. The motor 200 may be connected directly to the drive shaft 18 which in turn is engaged with a cutting cap 14 to confer a reciprocating motion to the cutting cap 14. In one aspect, the drive shaft 18 may be rotated by the motor 200. In another aspect, the drive shaft 18 may be reciprocated by the motor 200 or apparatus associated with the motor 200. The housing 100, as illustrated, contains the motor 200, drive shaft 18, and/or other apparatus for conferring a reciprocating movement to the cutting cap 14. Further as illustrated, housing 100 may be configured as a handle to permit a physician to position the guide tube 12 within a patient and/or operate the cutting mechanism. As such, the housing 100 may include apparatus for initiating and stopping the reciprocating movement of the cutting cap 14, such as, for example, electrical switches, mechanical switches, clutches, and the like. The guide tube 12 may be attached to the housing 100. The housing 100 may include a trigger 102 to turn on the motor 200. An actuator 104 may be positioned on a side of the housing 100 opposite the trigger 102. For exemplary purposes, the actuator 104 is illustrated as slidably secured within the housing 100. The actuator 104 may be operably connected to the motor 200 to slide the motor 200, illustrated in phantom, forward and backwards within the housing 100 as indicated by the arrows. In other embodiments, actuator 104 may be connected to guide tube 12 to extend and retract of the cutting cap 14 and cutting member 16 from the distal end of an outer guide tube 20.

Guide tube 12 may be secured to the distal end of housing 100. Guide tube 12 defines a lumen 22. Generally, lumen 22 contains a drive shaft 18 to confer a reciprocating movement to cutting cap 14. Lumen 22 may further be operably connected to a vacuum apparatus, not shown, to provide suction through lumen 22 to a distal opening 32 at the distal end of guide tube 12. Suction may be used to assist in removal tissue fragments from the nucleus. In addition or alternatively, suction may assist in the removal of tissue cut by cutting cap 14 by urging the tissue toward the cutting surface 46 of the cutting cap 14. Guide tube 12 may be configured from a material which permits a surgeon to properly position the distal portion of the guide tube 12 within an intervertebral disc to remove the desired portions of the intervertebral disc. In one aspect, applications may required that the guide tube 12 have sufficient flexibility to bend and otherwise flex as the distal end of the guide tube 12 is inserted through a patient into the intervertebral disc. In other aspects, applications may require that the guide tube 12 have sufficient stiffness to permit a surgeon to advance the distal end into the intervertebral disc and to precisely maneuver the distal portion of the guide tube 12 within the intervertebral disc. In still other aspects, the guide tube 12 may have a variable stiffness along its length for applications requiring or benefiting from such variable stiffness. In still other aspects, the guide tube 12 may be configured to follow the curves within the lumen 34 of an outer guide tube 20.

Typically, the material used for the guide tube 12 is polymeric such as a high density polyethylene, PTFE, PEBAX, PEEK or other flexible polymeric material which will be recognized by those skilled in the art. However, the material may be a metal, composite materials or other material selected and configured for access to the intervertebral disc. Alternatively, the guide tube 12 may be configured from a stiff material such as a metal to allow precise positioning and movement of the cutting member 16.

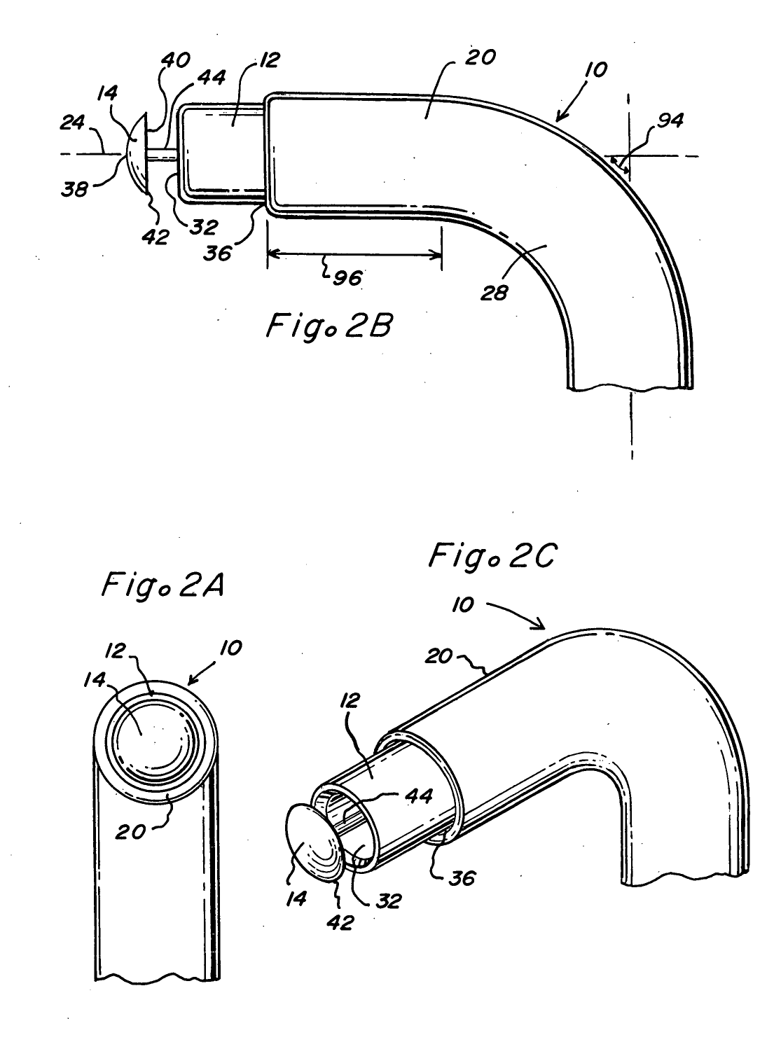

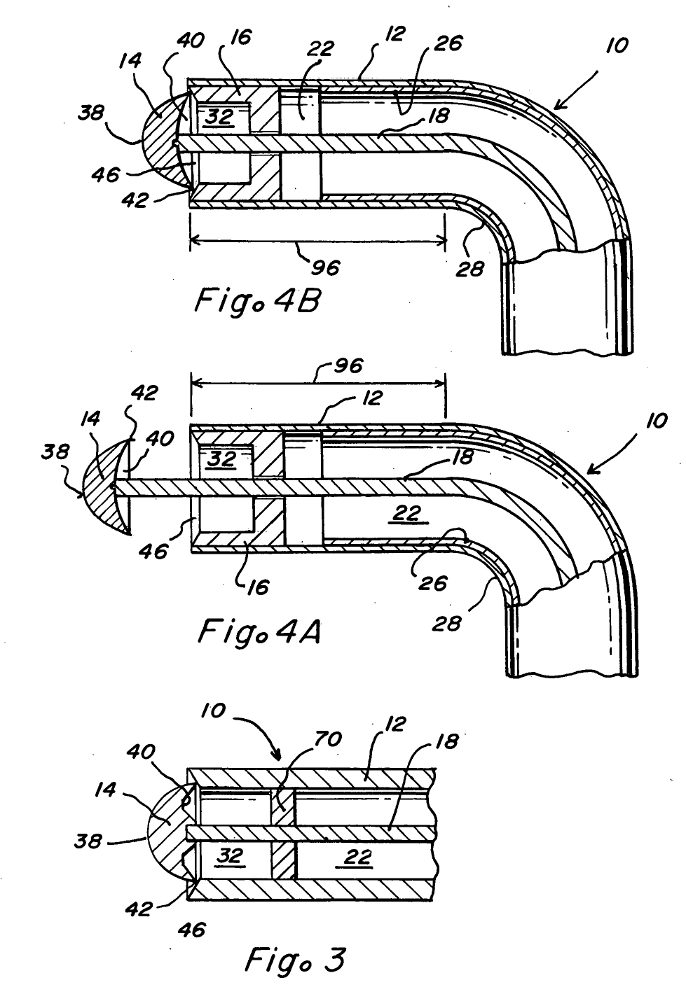

As illustrated throughout the Figures, the guide tube 12 defines lumen 22 that may extend along the longitudinal axis 24 of the guide tube 12. Longitudinal axis 24 may be curvilinear over portions of its length. In one aspect, the lumen 22 may include a lubricious coating 26, shown in FIGS. 4A and 4B, to reduce friction between the walls of lumen 22 and the drive shaft 18 or other components positioned within the lumen 22. A proximal end of guide tube 12 defines a proximal opening of the lumen 22. The proximal end of guide tube 12 may be adapted to engage a handle or housing 100, a motor 200 and/or other components associated with a reciprocating cutting apparatus 10. A distal end of guide tube 12 defines a distal opening 32 of the lumen 22. In one aspect, a bend 28 may be permanently formed and located near the distal end of the guide tube 12. Bend 28 may be straightened to varying degrees by external forces to which guide tube 12 may be subjected but will typically resume the bent configuration when these forces are removed. The bend 28 directs the guide tube 12 and the associated lumen 22 laterally at a desired angle 94 from the longitudinal axis 24. The angle 94 is typically between about sixty (60) degrees and one hundred twenty (120) degrees from the longitudinal axis 24. In one aspect, the angle 94, shown in FIG. 2, of the permanent bend 28 may be about ninety (90) degrees from the longitudinal axis 24 as is generally illustrated in the figures for exemplary purposes. In other aspects, guide tube 12 may be steerable. One method of providing a steerable feature is for guide tube 12 to possess a second, smaller lumen within the wall of guide tube 12 positioned along the outer radius of the permanent bend 28. A stiff rod or wire can be slidably moved within the smaller lumen, with the effect of straightening the permanent bend 28, at least partially, when the stiff rod or wire is fully inserted along the length of guide tube 12. In this aspect, the degree of bending can be controlled by a user and may be varied during the use of the reciprocating cutting apparatus 10. The lumen 22 and bend 28 are configured to generally direct the cutting action of the reciprocating cutting cap 14 and cutting member 16 laterally from the proximal portion of longitudinal axis 24. In one aspect, the distal end of guide tube 12 is configured to include linear section 96 of lumen 22 extending laterally between the bend 28 and the distal opening 32. In certain embodiments, the linear section 96 may permit a surgeon to orient and linearly advance the cutting cap 14 and cutting member 16 through the material of the intervertebral disc in a desired direction. In applications for extracting materials from an intervertebral disc, the linear section 96 of guide tube 20 is typically between 0.5 millimeters and 20 millimeters in length.

As illustrated in FIGS. 1 to 2C, 5A to 5C, 7A to 8B, guide tube 12 may be received within an outer lumen 34 of an outer guide tube 20. When an the outer guide tube 20 is utilized, the guide tube 12 may be slidably received within the outer lumen 34 of the outer guide tube 20 to permit the extending and retracting of the cutting cap 14 and cutting member 16 relative to the outer distal opening 36 of the outer lumen 34. The guide tube 12 may be extended or retracted within the outer guide tube 20 to extend or retract the cutting cap 14 and cutting member 16 from the outer distal opening 36 of outer guide tube 20.

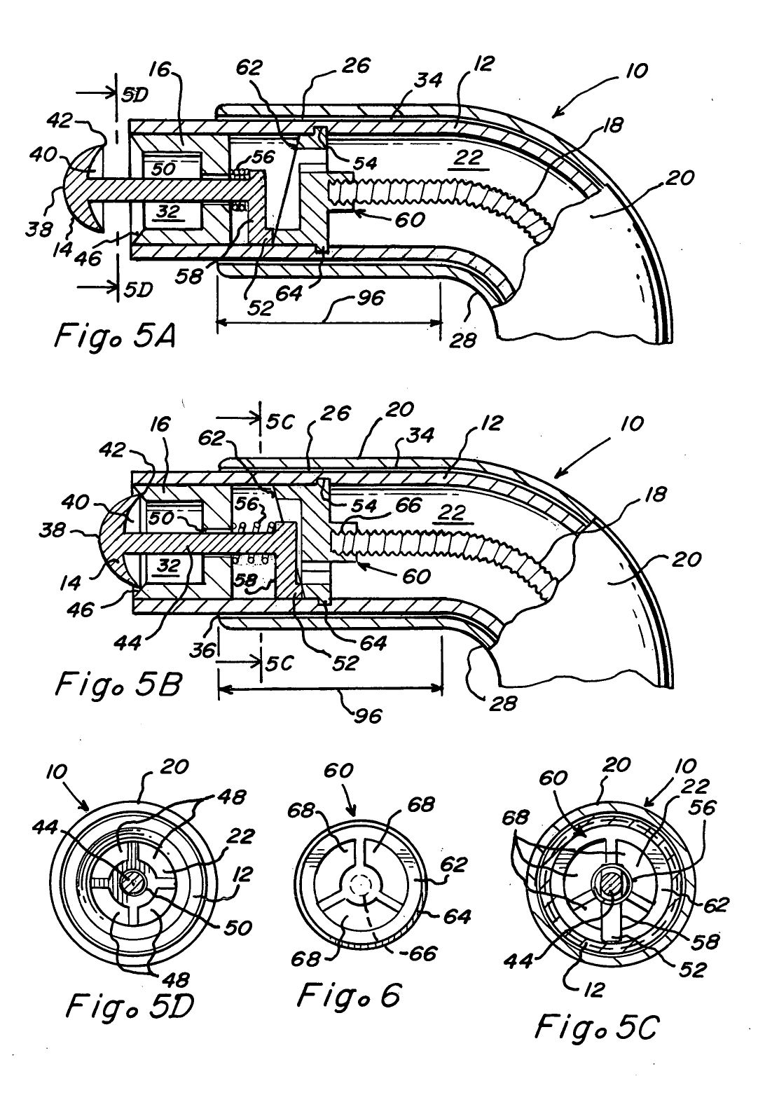

In these embodiments, the guide tube 12 is typically more flexible to accommodate following the outer lumen 34 of outer guide tube 20. The guide tube 12 may include a lubricious coating 26, shown in FIGS. 5A and 5B, on an exterior surface to facilitate sliding of the guide tube 12 within the outer lumen 34 of outer guide tube 36. When present, the outer lumen 34 of outer guide tube 20 may extend along the longitudinal axis 24 of outer guide tube 20. Longitudinal axis 24 may be curvilinear over portions of its length. A proximal end of outer guide tube 20 defines a proximal opening of the outer lumen 34. The proximal end of outer guide tube 20 may be adapted to engage a handle or housing 100, a motor 200 and/or other components associated with a reciprocating cutting apparatus 10. The guide tube 12 may communicate with the actuator 104 of the housing 100 to allow a user to extend or retract the cutting cap 14 and cutting member 16 from the outer distal opening 36. Drive shaft 18 may extend through the outer lumen 34 defined by the outer guide tube 20. As illustrated, outer lumen 34 may be generally coextensive with lumen 22.

A bend 28 may be located near the distal end of the outer guide tube 20. Bend 28 may be straightened to varying degrees by external forces to which outer guide tube 20 may be subjected but will typically resume the bent configuration when these forces are removed. The bend 28 directs the outer guide tube 20 and the associated guide tube 12 positioned within outer lumen 34 laterally at a desired angle 94 from the longitudinal axis 24. The angle 94 is typically between about sixty (60) degrees and one hundred twenty (120) degrees from the longitudinal axis 24. In one aspect, the angle 94, shown in FIG. 2, of the permanent bend 28 may be about ninety (90) degrees from the longitudinal axis 24 as is generally illustrated in the figures for exemplary purposes. In other aspects, outer guide tube 20 may be steerable. One method of providing a steerable feature is for outer guide tube 20 to possess a second, smaller lumen within a wall of outer guide tube 20 positioned along the outer radius of the permanent bend 28. A stiff rod or wire can be slidably moved within the smaller lumen, with the effect of straightening the permanent bend 28, at least partially, when the stiff rod or wire is fully inserted along the length of outer guide tube 20. In this aspect, the degree of bending can be controlled by a user and may be varied during the use of the reciprocating cutting apparatus 10. The outer lumen 34 and bend 28 of outer guide tube 20 are generally configured to direct the cutting action of reciprocating the cutting cap 14 associated with guide tube 20 laterally from the proximal portion of longitudinal axis 24. In one aspect, the distal end of outer guide tube 20 is configured to include linear section 96 of outer lumen 34 extending laterally between the permanent bend 28 and the outer distal opening 36. In certain embodiments, the linear section 96 of outer guide tube 20 may permit a surgeon to orient and linearly advance the cutting cap 14 and cutting member 16 at the distal end of guide tube 20 through the material of the intervertebral disc in a desired direction. In applications for extracting materials from an intervertebral disc, the linear section 96 of outer guide tube 20 is typically between 0.5 millimeters and 20 millimeters in length.

The drive shaft 18 may extend through at least a portion of lumen 22 and may extend through a least a portion of outer lumen 34. A distal end of the drive shaft 18 is connected to the cutting cap 14 to confer a reciprocating motion upon the cutting cap 14. The drive shaft 18 is typically operably connected to a motor 200 at a proximal end of the drive shaft 18. However, the drive shaft 18 may be otherwise connected to the motor 200 to confer a rotational or reciprocating motion upon the drive shaft 18 as will be recognized by those skilled in the art upon review of the present disclosure. The drive shaft 18 operably couples a motive component, such as for example a motor 200, conferring rotational or reciprocating movement to the cutting cap 14. A drive shaft 18 may, typically at a proximal end, be engaged with the motor 200, a transmission and/or clutch assembly connected to a motor 200, or to another rotational or reciprocal motivating component to confer a rotational or reciprocal force to a cutting cap 14. The drive shaft 18 are typically metals however a range of polymers and other materials may be used as will be recognized by those skilled in the art upon review of the present disclosure. Drive shaft 18 is frequently in the form of wires, cables, braided wires, coils, and tubes. In one aspect, the drive shaft 18 may define a driveshaft lumen such as may be the case when, for example, a coil is used as a drive shaft 18. A distal end of the drive shaft 18 typically engages the cutting cap 14. A drive shaft 18 in accordance with the present invention is typically of a diameter and configuration to be rotatably or reciprocatingly received within lumen 22 of guide tube 12. Typically, the drive shaft 18 will extend for a length greater than the length of the lumen 22. Such a length can permit the cutting cap 14 to be extended beyond the distal opening 32 of lumen 22 to engage a tissue within the intervertebral disc.

Cutting cap 14 is generally configured to cut, abrade or otherwise disrupt material to permit the concurrent or subsequent removal of tissue. Typically, cutting cap 14 is operably connected to the drive shaft 18 to reciprocate relative to the distal opening 32 of guide tube 20, an inner guide tube 20 and/or a cutting member 16. As illustrated in FIG. 2C, the cutting cap 14 may have a substantially circular shape and may be centered about axis 24. In one aspect, the crown 38 of the cutting cap 14 is configured to be atraumatic when brought into incidental contact with the annulus fibrosus as the cutting cap 14 is advanced through the nucleus pulposus. The crown 38 may be rounded, flattened, conical or otherwise configured to render such contact atraumatic as will be understood by those skilled in the art. A posterior surface 40 of cutting cap 14 is generally configured to cut or abrade the tissues of the intervertebral disc. Posterior surface 40 may be concave, flat or otherwise shaped. In one aspect, the posterior surface 40 may be configured with a trailing cutting edge 42 configured to cut or abrade the tissue of the nucleus pulposus. Trailing cutting edge 42 may be annular and peripherally positioned on cutting cap 14. Drive shaft 18 may be secured directly to the posterior surface 40 of cutting cap 14. Cutting cap 14 may further include a cap shaft 44, as shown in FIGS. 5A and 5B, extending from the posterior surface 40 which communicates, directly or indirectly, with drive shaft 18 to confer a reciprocating motion to cutting cap 14. In one aspect, cutting cap 14 may act in combination with cutting member 16 to cut, abrade or otherwise disrupt the material of the intervertebral disc. In another aspect, cutting cap 14 may act in combination with guide tube 12, as illustrated in FIG. 3, to cut, abrade or otherwise disrupt the material of the intervertebral disc. The material cut, abraded or disrupted may be limited to the tissue of the nucleus pulposus.

Cutting member 16 may be received within the proximal end of lumen 22 of guide tube 12 an end view of which is illustrated in isolation in FIG. 6. In another aspect, cutting member 16 may be secured to the distal end of guide tube 12. Cutting member 16 may be substantially tubular in shape and is typically sized to be received within either lumen 22 of guide tube 12 or outer lumen 34 of outer guide tube 20 or may be configured to have an outside diameter substantially equivalent to the outside diameter of the guide tube 12 or the outer guide tube 20 when secured to the distal end of the guide tube 12 or the outer guide tube 20, respectively. Cutting member 16 may define one or more passages 48. Passages 48 will typically communicate with at least one of lumen 22 and outer lumen 34 and may be configured to permit the passage of tissue fragments cut or abraded by cutting cap 14. Cutting member 16 may also define a guide 50. Guide 50 may slidably receive either the cap shaft 44 or the drive shaft 18. Cutting member 16 may define a cutting surface 46 to receive the posterior surface 40 of cutting cap 14 to assist in cutting or abrading of tissue. When a cutting member 16 is not provided, a cutting surface 46 may be defined by a surface of guide tube 12 about the distal opening 32. In one aspect, cutting surface 46 may be annular. In another aspect, cutting surface 46 may receive the trailing cutting edge 42 of cutting cap 14.

FIG. 3 illustrates an exemplary embodiment of a reciprocating cutting apparatus 10 without a cutting member 16. Cutting cap 14 reciprocates relative to the distal end of guide tube 12 which defines a cutting surface 46 which may receive the trailing cutting edge 42 of cutting cap 14 when the cutting cap is in the withdrawn position. One or more independent shaft guides 70 may be provided to slidably or rotatably receive at least one of the cap shaft 44 and the drive shaft 18. Shaft guides 70 may be positioned at the distal end of the lumen 22 and/or at various other locations along the length of lumen 22.

FIGS. 4A and 4B illustrate an exemplary embodiment of a reciprocating cutting apparatus 10 in accordance with the present invention. The embodiment of FIGS. 4A and 4B has a reciprocating drive shaft 18 secured directly to a cutting cap 14. The cutting cap 14 extends from a distal opening 32 in guide tube 12. FIG. 4A illustrates the cutting cap 14 in an extended position and FIG. 4B illustrates cutting cap 14 in a retracted position. The crown 38 of cutting cap 14 is rounded for exemplary purposes which can render contact with the relatively tough annulus fibrosus substantially atraumatic as the cutting cap 14 is reciprocating relative to the distal opening 32 in guide tube 12. Further, the rounded crown 38 of cutting cap 14 may permit the cutting cap 14 to be driven through the relatively soft tissue of the nucleus pulposus on the extension stroke of its reciprocating movement and as the reciprocating cutting apparatus 10 is advanced by a physician. The posterior surface 40 of cutting cap 14 has a concave surface and includes a trailing cutting edge 42. Cutting member 16 is secured within the distal end of lumen 22 adjacent to the distal opening 32.

The cutting member 16, as illustrated in FIGS. 4A and 4B, includes a beveled cutting surface 46. The guide tube 20, as illustrated in FIG. 3, includes a beveled cutting surface 46 positioned about distal opening 32. The cutting surface 46 is beveled inward to assist in directing materials into the lumen 22. The trailing cutting edge 42 may contact the cutting surface 46 of the cutting member 16 when the cutting cap 14 is fully retracted. In one aspect, this contact may cause tissue positioned between the trailing cutting edge 42 and cutting surface 46 to be cut from the bulk material of a patient's nucleus pulposus. Further, the concave posterior surface 40 of the cutting cap 14 may tend to receive and maintain cut tissue and direct it to the distal opening 32 of guide tube 12. The guide tube 12 of the apparatus of FIGS. 4A and 4B includes a lubricious coating 26 to more easily permit the rotation of drive shaft 18 within lumen 22. The cutting member 16 includes a guide 50 through which drive shaft 18 is slidably positioned. The guide 50 may maintain cutting cap 14 concentric with cutting member 16 as the cutting cap 14 rapidly reciprocates between an extended and a retracted position and as forces are conferred upon the cutting cap 14 and guide tube 12 as the reciprocating cutting apparatus 10 is advanced and as it cuts tissue.

FIGS. 5A to 5D and 6 illustrate another exemplary embodiment of a reciprocating cutting apparatus 10 in accordance with the present invention. The embodiment of FIGS. 5A and 5B has a rotating drive shaft 18 secured directly to a cam 60 to confer a reciprocating motion upon cutting cap 14. The drive shaft 18 is illustrated as a coiled material for exemplary purposes. Further, the embodiment of FIGS. 5A and 5B include a guide tube 12 positioned within outer lumen 34 of outer guide tube 20. FIG. 6 illustrates an end view of the cam 60 alone showing the peripheral flange 64 and cam surface 62 extending about the diameter of the cam 60.

The cam 60 may be rotatably secured at a desired position within the lumen 22 of guide tube 12. As illustrated for exemplary purposes in FIGS. 5A to 5D and 6, cam 60 includes a peripheral flange 64 that is received in a circumferential groove 54 within the lumen 22 of guide tube 12 to rotatably secure cam 60 within lumen 22. The peripheral flange 64 may extend around at least a portion of the periphery of cam 60. Drive shaft 18 is typically concentrically secured to cam 60 to impart a rotational movement to cam 60. One or more cam passages 68 may extend longitudinally through the cam 60. Cam passages 68 may communicate with at least one of lumen 22 and outer lumen 34 and may be configured to permit the passage of tissue fragments cut or abraded by cutting cap 14. The cam 60 includes a cam surface 62. As illustrated cam surface 62 extends circumferentially about the periphery of cam 60 for exemplary purposes. Cap shaft 44 is connected to or integral with a cam follower 52 which contacts the cam surface 62 of cam 60. A cam support 58 may be secured to cap shaft 44 to connect the cam follower 52 to the cap shaft 44. A spring 56 or other resilient member may be provided to bias the cutting cap 14 in a retracted or an extended position. As illustrated, the spring 56 may be circumferentially secured about a proximal portion of cap shaft 44. Particularly as illustrated, spring 56 may be in contact, at a first end, with a posterior surface 40 of the cutting member 16 and, at a second end, with a cam support 58 for cam follower 52. Accordingly as illustrated, as cam 60 is rotated by the rotation of drive shaft 18, cam follower 52 follows the profile of cam surface 62 which causes cam follower 52 and the associated cap shaft 44 to reciprocate. Accordingly, cutting cap 14 is caused to move between an extended and a retracted position. Upon review of the present disclosure, those skilled in the art will recognize other cam/follower configurations to impart a linear reciprocating movement to a cutting cap 14 without departing from the scope of the present invention.

In some embodiments, the guide tube 12 may be extended from and retracted into the outer distal opening 36 in outer guide tube 20. A physician can extend the guide tube 12 relative to the outer distal opening 36 of the outer guide tube 20 to advance the reciprocating cutting cap 14 through the nucleus pulposus of an intervertebral disc. FIG. 5A illustrates the cutting cap 14 in an extended position and FIG. 5B illustrates cutting cap 14 in a retracted position. The crown 38 of cutting cap 14 is again rounded which can render contact with the relatively tough annulus fibrosus substantially atraumatic as the cutting cap 14 is reciprocating relative to the inner guide tube 20. Further, the rounded crown 38 of cutting cap 14 may permit the cutting cap 14 to be driven through the relatively soft tissue of the nucleus pulposus on the extension stroke of its reciprocating movement and as the apparatus is advanced by a physician. The posterior surface 40 of cutting cap 14 has a concave surface and includes a trailing cutting edge 42. Cutting member 16 is secured within the distal end of lumen 22 adjacent to the distal opening 32. The cutting member 16, as illustrated, includes a beveled cutting surface 46. The cutting surface 46 may be beveled inward. In one aspect, this may direct cut materials into lumen 22. The trailing cutting edge 42 may contact the cutting surface 46 of the cutting member 16 when the cutting cap 14 is fully retracted. In one aspect, this contact may cause tissue positioned between the trailing cutting edge 42 and cutting surface 46 to be cut from the bulk material of a patient's nucleus pulposus. Further, the concave posterior surface 40 of the cutting cap 14 may tend to receive and maintain cut tissue and direct it to the distal opening 32 of guide tube 12. The guide tube 12 of FIGS. 5A to 5D includes a lubricious coating 26 to more easily permit the guide tube 12 to slide within the outer lumen 34. The cutting member 16 may include a guide 50 through which cap shaft 44 is rotatably positioned. The guide 50 may maintain cutting cap 14 concentric with cutting member 16 as the cutting cap 14 rapidly reciprocates between an extended and a retracted position and as forces are conferred upon the cutting cap 14 and guide tube 12 as the reciprocating cutting apparatus 10 is advanced and as it cuts tissue.

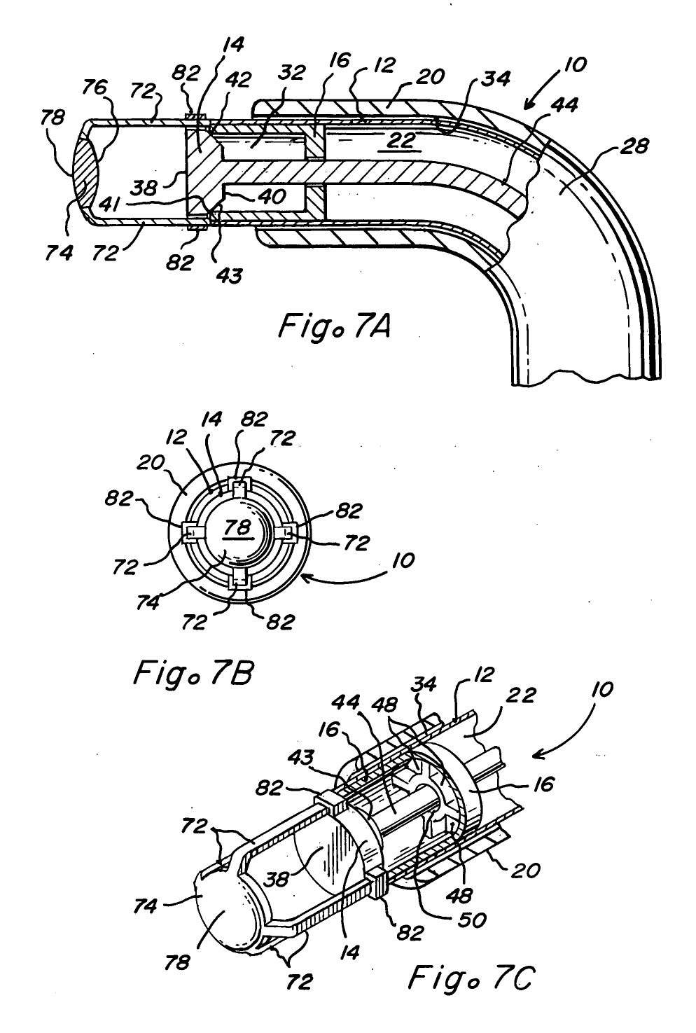

FIGS. 7A, 7B and 7C illustrate another exemplary embodiment of a reciprocating cutting apparatus 10 in accordance with the present invention. The embodiment of FIGS. 7A, 7B and 7C has a reciprocating cap shaft 44 secured directly to a cutting cap 14 and a distal stop 74. FIGS. 7A and 7C generally illustrate cutting cap 14 in a retracted position. Further, the embodiment of FIGS. 7A, 7B and 7C include a guide tube 12 positioned within outer lumen 34 of outer guide tube 20. The guide tube 12 may be extended from and retracted into the outer distal opening 36 in outer guide tube 20. A physician can extend the guide tube 12 relative to the outer distal opening 36 of the outer guide tube 20 to advance the distal stop 74 and proximally positioned reciprocating cutting cap 14 through the nucleus pulposus of an intervertebral disc.

As illustrated in FIGS. 7A, 7B and 7C, the cutting cap 14 extends from a distal opening 32. The distal stop 74 is secured to at least one of the cutting member 16 and the guide tube 12. Typically, distal stop 74 will include at least one stop support 72 to position distal stop 74 relative to the cutting cap 14. As illustrated, distal stop 74 includes a plurality of stop supports 72. Stop supports 72 may be secured to or integral with the distal stop 74 at their distal ends. At their proximal ends, stop supports 72 may be secured to or integral with the distal end of at least one of the cutting member 16 and the guide tube 12. The distal crown 78 of distal stop 74 is rounded for exemplary purposes. In operation, the distal crown 78 may form the leading edge of the reciprocating cutting apparatus 10 as the distal end of the reciprocating cutting apparatus 10 is advanced through the nucleus pulposus. The rounded configuration of distal crown 78 may render any incidental contact of distal stop 74 with the annulus fibrosus substantially atraumatic.

Generally, the cutting cap 14 reciprocates between the distal stop 74 and a distal opening 32. As illustrated, the cutting cap 14 reciprocates between a proximal surface 76 of distal stop 74 and a distal opening 32 of the guide tube 12. In one aspect, the proximal surface 76 of distal stop 74 may contact the crown 38 of cutting cap 14 when the cutting cap 14 is in a fully extended position or is approaching a fully extended position. In the embodiment illustrated in FIGS. 7A, 7B and 7C, the crown 38 of cutting cap 14 does not necessarily have to be atraumatic to the annulus fibrosus because crown 38 does not form the leading edge of a reciprocating cutting apparatus 10 as the distal portion of reciprocating cutting apparatus 10 is advanced through the nucleus pulposus.

Cutting member 16 is secured to the distal end of lumen 22 adjacent to the distal opening 32. As illustrated for exemplary purposes, the cutting member 16 is secured within the distal end of lumen 22. The cutting member 16 can include a guide 50 through which a cap shaft 44 or drive shaft 18 may be slidably or rotatably positioned. The guide 50 may maintain cutting cap 14 concentric with cutting member 16 as the cutting cap 14 rapidly reciprocates between an extended and a retracted position and as forces are conferred upon the cutting cap 14 and guide tube 12 as the reciprocating cutting apparatus 10 is advanced and as it cuts tissue. As illustrated, the posterior surface 40 of cutting cap 14 includes a beveled edge 43 and cutting member 16 includes a cutting edge 41. The cutting edge 41 of the cutting member 16 may contact the beveled edge 43 of cutting cap 14 when the cutting cap 14 is fully retracted. In one aspect, the juxtaposition or contact of beveled edge 43 of cutting cap 14 and the cutting edge 41 of cutting member 16 may cut tissue when the cutting cap 14 is in or is approaching a retracted position.

FIGS. 8A and 8B illustrate yet another exemplary embodiment of a reciprocating cutting apparatus 10 in accordance with the present invention. The embodiment of FIGS. 8A and 8B has a reciprocating cap shaft 44 secured directly to a cutting cap 14 and a distal stop 74. The cap shaft 44 is also integral with the drive shaft 18 for exemplary purposes. FIG. 8A generally illustrates cutting cap 14 in a retracted position and FIG. 8B generally illustrates cutting cap 14 in an extended position. The proximal surface 76 of distal stop 74 is configured to receive a leading cutting edge 45 of the cutting cap 14 when the cutting cap 14 is in an extended position. Further, the embodiment of FIGS. 8A and 8B again include a guide tube 12 positioned within outer lumen 34 of outer guide tube 20. The guide tube 12 may be extended from and retracted into the outer distal opening 36 in outer guide tube 20. A physician can extend the guide tube 12 relative to the outer distal opening 36 of the outer guide tube 20 to advance the distal stop 74 and proximally positioned reciprocating cutting cap 14 through the nucleus pulposus of an intervertebral disc.

As illustrated in FIGS. 8A and 8B, the cutting cap 14 extends from a distal opening 32. A distal stop 74 is again secured to the distal end of at least one of the cutting member 16 and the guide tube 12. Typically, distal stop 74 includes at least one stop support 72 to position distal stop 74 relative to the cutting cap 14. As illustrated, distal stop 74 includes a plurality of stop supports 72. Stop supports 72 may be secured to or integral with the distal stop 74 at their distal ends. At their proximal ends, stop supports 72 may be secured to or integral with the distal end of at least one of the cutting member 16 and the guide tube 12. In one aspect, cutting cap 14 may include one or more cap guides 82 positioned about its periphery. The cap guides 82 may be configured as grooves in the cutting cap 14 or may be guides defining channels or passages to slidably receive stop supports 72. Each cap guides 82 may be configured to slidably receive a stop support 72 as the cutting cap 14 reciprocates between an extended and a retracted position. In one aspect, cap guides 82 slidably receiving the stop supports 72 may permit consistent relative positioning of the distal stop 74 and cutting cap 14 when the cutting cap 14 is in the extended position.

The cutting cap 14 of the embodiment illustrated in FIGS. 8A and 8B includes both a trailing cutting edge 42 and a leading cutting edge 45. The trailing cutting edge 42 is generally configured to cut and/or abrade tissue as the cutting cap 14 is withdrawn into lumen 22 similar to the cutting and/or abrading action of the embodiments illustrated without a leading cutting edge 45. The leading cutting edge 45 is generally configured to cut and/or abrade tissue as the cutting cap 14 is extended from the lumen 22.

The distal crown 78 of distal stop 74 is again generally configured to be atraumatic upon incidental contact with the annulus fibrosus. Again for exemplary purposes, the distal crown 78 has been illustrated as rounded for exemplary purposes. Those skilled in the art will recognize additional configurations for distal crown 78 that may render any incidental contact of distal stop 74 with the annulus fibrosus substantially atraumatic upon review and understanding of the inventions of the present disclosure. The proximal surface 76 of distal stop 74 is configured to cooperate with the leading cutting edge 45 of cutting cap 14 in the cutting and/or abrading of tissue. In one aspect, the proximal surface 76 of distal stop 74 may contact the leading cutting edge 45 of cutting cap 14 when the cutting cap 14 is in a fully extended position or is approaching a fully extended position.

Cutting member 16 is secured to the distal end of lumen 22 adjacent to the distal opening 32. As illustrated for exemplary purposes, the cutting member 16 is secured within the distal end of lumen 22. The cutting member 16 can include a guide 50 through which a cap shaft 44 or drive shaft 18 may be slidably positioned. The guide 50 may maintain cutting cap 14 concentric with cutting member 16 as the cutting cap 14 rapidly reciprocates between an extended and a retracted position and as forces are conferred upon the cutting cap 14 and guide tube 12 as the reciprocating cutting apparatus 10 is advanced and as it cuts tissue. The cutting member 16, as illustrated, includes a beveled cutting surface 46. The cutting surface 46 is beveled inward which may permit the guiding of cut and/or abraded debris into the distal opening 32.

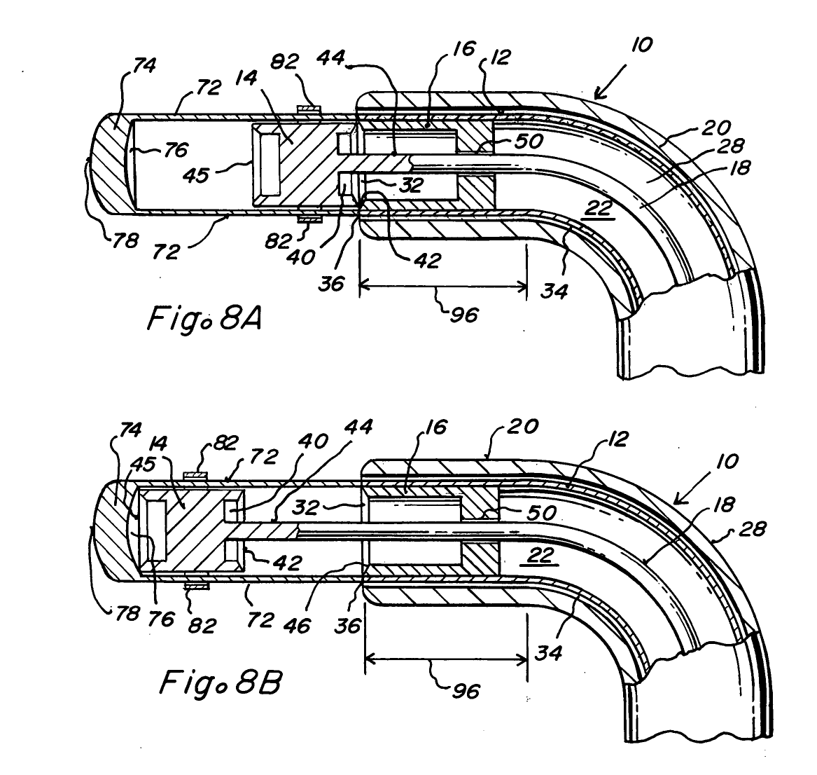

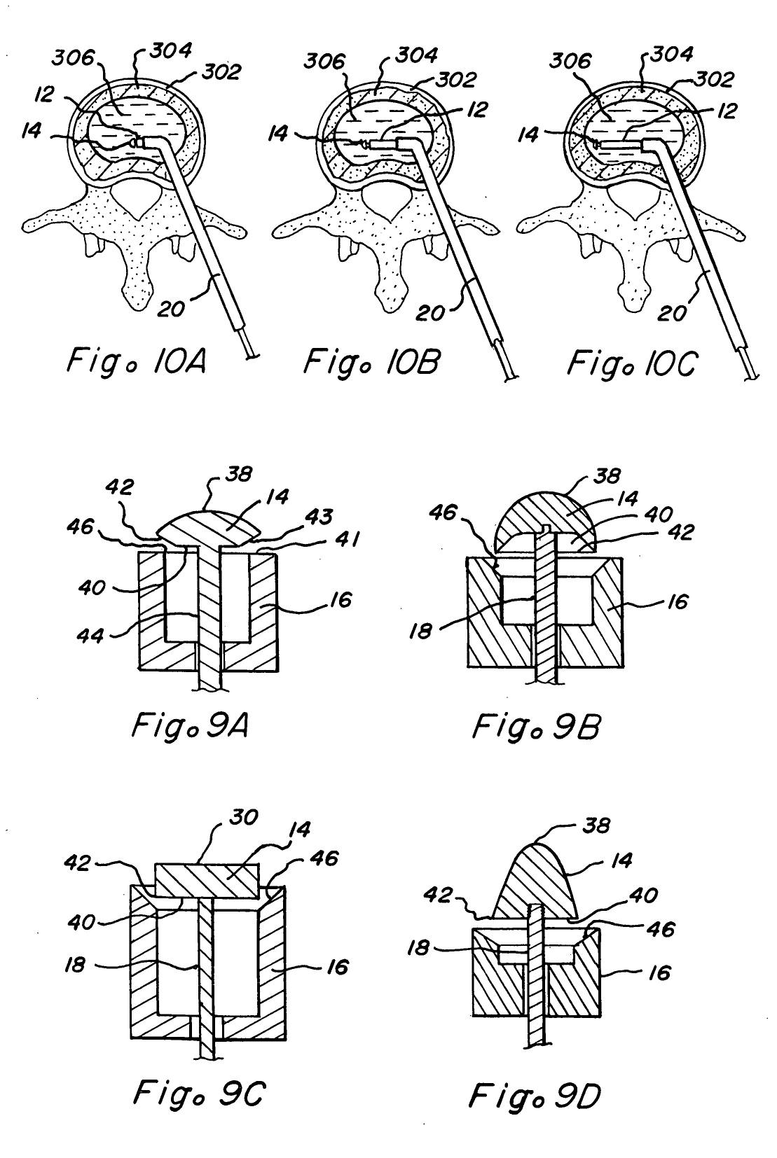

FIGS. 9A to 9D illustrate exemplary designs for the interaction of cutting cap 14 and cutting member 16 which may also be applicable to designs for cutting surface 46 formed in the distal end of guide tube 12 about the distal opening 32. FIG. 9A illustrates a cutting cap 14 having a crown 38 with an arcuate shape to be atraumatic to the annulus fibrosus during operation of reciprocating cutting apparatus 10. Trailing cutting edge 42 is beveled edge 43 between the crown 38 and the posterior surface 40. The cutting surface 46 is the cutting edge 41 formed by the transition between the distal end of cutting member 16 and the lumen defined through the cutting member 16. The illustrated posterior surface 40 is substantially planar. FIG. 9B illustrates a cutting cap 14 having a crown 38 with a semi-circular shape to be atraumatic to the annulus fibrosus during operation of reciprocating cutting apparatus 10. Trailing cutting edge 42 is a circumferential lip between the semicircular crown 38 and a concave posterior surface 40. The cutting surface 46 is beveled to form an angled surface. FIG. 9C illustrates a cutting cap 14 having a crown 38 with a substantially planar shape to be atraumatic to the annulus fibrosus during operation of reciprocating cutting apparatus 10. Trailing cutting edge 42 is a circumferential edge between a side of cutting cap 14 and a substantially planar posterior surface 40. The cutting surface 46 is again illustrated with a bevel to form an angled surface. FIG. 9D illustrates a cutting cap 14 having a crown 38 with a substantially conical shape to be atraumatic to the annulus fibrosus during operation of reciprocating cutting apparatus 10. Trailing cutting edge 42 is a circumferential edge between crown 38 and a substantially planar posterior surface 40. The cutting surface 46 is again illustrated with a bevel to form an angled surface. These provide some exemplary configurations which can be atraumatic to the annulus fibrosus and can facilitate the cutting and/abrading of the nucleus pulposus as the cutting cap 14 is advanced through the nucleus pulposus.

In operation, the cutting cap 14 reciprocates between the proximal surface 76 of distal stop 74 in an extended position and a distal opening 32 in a retracted position. As the cutting cap 14 is withdrawn, the trailing cutting edge 42 may cut and/or abrade tissue. When the trailing cutting edge 42 is fully retracted, the trailing cutting edge 42 of the cutting cap 14 and the cutting surface 46 of the cutting member 16 are brought into contact and/or close proximity to one another. In one aspect, this contact and/or proximity may cause tissue positioned between the trailing cutting edge 42 and cutting surface 46 to be cut from the bulk material of a patient's nucleus pulposus. Further, the cavity in the posterior surface 40 of the cutting cap 14 may tend to receive and maintain cut tissue and direct it to the distal opening 32 of guide tube 12. As the cutting cap 14 is extended, the leading cutting edge 45 may also tend to cut and/or abrade tissue. When the leading cutting edge 45 is fully extended, the leading cutting edge 45 of the cutting cap 14 and the proximal surface 76 of the distal stop 74 are brought into contact and/or close proximity to one another. In one aspect, this contact and/or proximity may cause tissue positioned between the leading cutting edge 45 and the proximal surface 76 of the distal stop 74 to be cut from the bulk material of a patient's nucleus pulposus.

FIGS. 10A, 10B and 10C illustrate an exemplary sequence and methodology for advancing the distal end of guide tube 12 and associated cutting cap 14 through a nucleus pulposus 306 of an intervertebral disc 302 in a de-nucleating procedure. FIG. 10A illustrates the outer guide tube 20 positioned and oriented within the nucleus pulposus 306 of an intervertebral disc with the guide tube 12 and associated cutting cap 14 retracted within the outer guide tube 20. FIG. 10B illustrates the guide tube 12 and associated cutting cap 14 advancing through the nucleus pulposus 306 of the intervertebral disc as the guide tube 12 is extended from outer lumen 34 of outer guide tube 20. As guide tube 12 is advanced, the associated cutting cap 14 reciprocates relative to the guide tube 12 and the distal opening 32 to cut and/or abrade the tissue of the nucleus pulposus 306. FIG. 10C illustrates the guide tube 12 in an extended position with the cutting cap 14 having cut a substantially straight track across the nucleus pulposus 306 and atraumatically contacting the annulus fibrosus 304 located about the periphery of the intervertebral disc 302. Once the guide tube 12 has been extended as far as desired, which may be to the periphery of the annulus fibrosus 304, the guide tube 12 and associated cutting cap 14 are retracted into the outer guide tube 20. More tissue can be removed by advancing the guide tube 20 further ventrally into the disc cavity and repeating the steps shown in FIGS. 10A to 10C. The nucleus pulposus 306 along the opposite side of the outer guide tube 20 can be removed by rotating the outer guide tube 20 one hundred eighty (180) degrees about its long axis and repeating the steps shown in FIGS. 10A to 10C while step-wise advancing or withdrawing the guide tube 12 from the disc cavity.

The foregoing discussion discloses and describes merely exemplary embodiments of the present invention. Upon review of the specification, one skilled in the art will readily recognize from such discussion, and from the accompanying drawings and claims, that various changes, modifications and variations can be made therein without departing from the spirit and scope of the invention as defined in the following claims.

What is claimed is:

1. A reciprocating cutting apparatus for removing tissue from an intervertebral disc, comprising:
a guide tube defining a lumen extending through the guide tube from a proximal opening at a proximal end of the guide tube to a distal opening at a distal end of the guide tube, the lumen including a bend at the distal end of the guide tube, the lumen of the guide tube extending linearly over a linear section extending between the bend and the distal opening;
a drive shaft received within the lumen of the guide tube, the drive shaft having a proximal end and a distal end; and
a cutting cap defining a trailing cutting edge and an atraumatic crown, the drive shaft operably connected to the cutting cap to confer a reciprocating motion to the cutting cap, the cutting cap movable between an extended position and a retracted position relative to the distal opening of the guide tube.

2. An apparatus, as in claim 1, further comprising the cutting cap secured to a distal end of the drive shaft.

3. An apparatus, as in claim 1, further comprising a cutting surface on a distal end of the guide tube to receive the trailing cutting edge of the cutting cap when the cutting cap is in a withdrawn position.

4. An apparatus as in claim 1, further comprising the cutting surface defined on a cutting member to receive the trailing cutting edge of the cutting cap when the cutting cap is in a withdrawn position, the cutting member secured to the guide tube.

5. An apparatus as in claim 4, further comprising the cutting member secured within the lumen of the guide tube at the distal opening of the lumen.

6. An apparatus, as in claim 1, further comprising the guide tube slidably received within an outer guide tube.

7. An apparatus, as in claim 1, further comprising a motor connected to a distal end of the drive shaft to confer a reciprocating motion to the drive shaft.

8. An apparatus, as in claim 1, further comprising:
a cam rotatably secured within the lumen of the guide tube, the cam defining a cam surface and having a shaft mount, the shaft mount secured to the drive shaft;
a cam follower biased against the cam surface to convert a rotation of the cam into a reciprocating motion; and
a cap shaft secured at a first end to the cam follower and at a second end to the cutting cap.

9. An apparatus, as in claim 8, further comprising the guide tube slidably received within an outer guide tube.

10. An apparatus, as in claim 8, further comprising a motor connected to a distal end of the drive shaft to confer a rotating motion to the drive shaft.

11. A reciprocating cutting apparatus for removing tissue from an intervertebral disc, comprising:
a guide tube defining a lumen extending through the guide tube from a proximal opening at a proximal end of the guide tube to a distal opening at a distal end of the guide tube, the lumen including a bend at the distal end of the guide tube, the lumen of the guide tube extending linearly over a linear section extending between the bend and the distal opening;
a distal stop secured to a distal end of the guide tube;
a drive shaft received within the lumen of the guide tube, the drive shaft having a proximal end and a distal end; and
a cutting cap defining a trailing cutting edge and a leading cutting edge, the drive shaft operably connected to the cutting cap to confer a reciprocating motion to the cutting cap, the cutting cap movable between an extended position and a retracted position relative to the distal opening of the guide tube.

12. An apparatus, as in claim 11, further comprising the cutting cap secured to a distal end of the drive shaft.

13. An apparatus, as in claim 11, further comprising at least one stop support extending between the distal end of the guide tube and the distal stop to secure the distal stop relative to the distal opening of the guide tube.

14. An apparatus, as in claim 13, further comprising at least one cap guide secured to the cutting cap and slidably receiving at least one of the stop supports.

15. An apparatus, as in claim 13, further comprising at least one cap guide integral with the cutting cap and slidably receiving at least one of the stop supports.

16. An apparatus, as in claim 11, further comprising a cutting surface on a distal end of the guide tube.

17. An apparatus as in claim 11, further comprising the cutting surface defined on a cutting member, the cutting member secured to the guide tube.

18. An apparatus as in claim 17, further comprising the cutting member secured within the lumen of the guide tube at the distal opening of the lumen.

19. An apparatus, as in claim 11, further comprising the guide tube slidably received within an outer guide tube.

20. An apparatus, as in claim 11, further comprising a motor connected to a distal end of the drive shaft to confer a reciprocating motion to the drive shaft.

21. An apparatus, as in claim 11, further comprising:
a cam rotatably secured within the lumen of the guide tube, the cam defining a cam surface and having a shaft mount, the shaft mount secured to the drive shaft;
a cam follower biased against the cam surface to convert a rotation of the cam into a reciprocating motion; and
a cap shaft secured at a first end to the cam follower and at a second end to the cutting cap.

22. An apparatus, as in claim 21, further comprising the guide tube slidably received within an outer guide tube.

23. An apparatus, as in claim 21, further comprising a motor connected to a distal end of the drive shaft to confer a rotating motion to the drive shaft.
